(19) United States
(12) Patent Application Publication
(10) Pub. No.: US 2023/0113107 A1
(43) Pub. Date: Apr. 13, 2023

(54) SENSING DEVICE

(71) Applicant: LG INNOTEK CO., LTD., Seoul (KR)

(72) Inventors: Do Hee KEUM, Seoul (KR); Kab Young KIM, Seoul (KR); Beom Sun HONG, Seoul (KR)

(73) Assignee: LG INNOTEK CO., LTD., Seoul (KR)

(21) Appl. No.: 17/911,321

(22) PCT Filed: Mar. 10, 2021
(86) PCT No.: PCT/KR2021/002966

§ 371 Date: Sep. 13, 2022

(30) Foreign Application Priority Data
Mar. 17, 2020 (KR) 10-2020-0032604

(51) Int. Cl. A61B 5/1486; A61B 5/00; A61B 5/145

(52) U.S. Cl. CPC A61B 5/14865; A61B 5/0031; A61B 5/14532; A61B 5/6847; A61B 2562/164; A61B 2562/227; A61B 2562/166

(57) ABSTRACT

A sensing device according to one embodiment of the present invention comprises: a substrate; a sensor that includes an electrode disposed on the substrate, and a connection terminal disposed on the substrate and connected to the electrode; and a stretchable substrate that is connected to the sensor unit and includes a base and wiring disposed on the base, wherein the connection terminal of the sensor unit is connected to the wiring of the stretchable substrate.

### TECHNICAL FIELD

The present invention relates to a sensing device, and more particularly, to a sensing device including an in-body sensor to be inserted into the body.

### BACKGROUND ART

With the development of medical technology, research on medical bioinformation measurement for monitoring body components and physiological information in real time is being actively conducted. Amid this, an interest in a sensing device for accurately measuring body components in real time is increasing.

As an example, a sensing device including an in-body sensor may have a structure in which a sensor coated with a bioreactive material that reacts with a body component in an interstitial fluid penetrates skin and is inserted into the human body, and an electrical signal generated due to an electrochemical action between the body component and the bioreactive material is transmitted to a signal processing unit disposed outside the body.

In this case, as a size of the in-body sensor inserted into the human body increases, a contact area with the body component may increase, and thus sensing accuracy may increase. However, as the size of the in-body sensor increases, a feeling of irritation felt by a user may increase.

In addition, other foreign materials such as proteins flowing in the interstitial fluid as well as the body component to be detected may be adsorbed onto the in-body sensor inserted into the human body. When foreign materials are adsorbed onto a sensor, sensing accuracy may be lowered, and a lifetime of the sensor may be shortened.

Meanwhile, a transmitter which receives, processes, and transmits a signal detected by the in-body sensor may be connected to the in-body sensor. In general, the transmitter may include a hard printed circuit board (PCB) accommodated in a hard material case, and when such a transmitter is attached to the skin, it may cause discomfort to the user.

### DISCLOSURE

### Technical Problem

The present invention is directed to providing a sensing device which is accurate, has a long lifetime, and minimizes a user's discomfort.

### Technical Solution

According to an embodiment of the present invention, a sensing device includes a sensor which includes a substrate, an electrode disposed on the substrate, and a connection terminal disposed on the substrate and connected to the electrode, and a stretchable substrate which is connected to the sensor and includes a base and a line disposed on the base, wherein the connection terminal of the sensor is connected to the line of the stretchable substrate.

The stretchable substrate may include a plurality of line layers which are stacked, and the connection terminal of the sensor may be disposed between two line layers among the plurality of line layers.

The base may be disposed between adjacent line layers among the plurality of line layers.

Each of the plurality of line layers may include a metal layer and a support layer.

At least a portion of the sensor may be inserted into the stretchable substrate, and the remaining portion thereof may be drawn out of the stretchable substrate.

The electrode of the sensor drawn out of the stretchable substrate may be accommodated in a biodegradable sensor guide and may be injected into a body together with the biodegradable sensor guide.

A portion of the sensor guide may be inserted into the stretchable substrate, and the remaining portion of the sensor guide may be exposed out of the stretchable substrate.

A length of the remaining portion of the sensor guide may be greater than a length of the portion of the sensor guide.

The substrate may be divided into an electrode region in which the electrode is disposed and a connection terminal region in which the connection terminal is disposed, and a width of the connection terminal region may be greater than a width of the electrode region.

The width of the connection terminal region may be greater than 1 times and not more than 5 times the width of the electrode region.

The line of the stretchable substrate may include a plurality of pads and a connector configured to connect the plurality of pads, and a width of the connection terminal may be different from a width of the pad.

The sensing device may further include an adhesive portion disposed between the connection terminal and the pad.

A width of the adhesive portion may be between the width of the connection terminal and the width of the pad.

The plurality of line layers may include a first line layer facing a first surface on which the connection terminal of the sensor is disposed among two surfaces of the substrate and a second line layer facing a second surface opposite to the first surface, and the sensing device may further include at least one of a signal processing unit and a transmission unit connected to the first line layer and buried in the stretchable substrate.

The at least one of the signal processing unit and the transmission unit may include a hard printed circuit board (PCB) and a chip disposed on the hard PCB.

A signal processing circuit pattern configured to process a signal received from the electrode through the connection terminal may be further disposed on a first surface on which the connection terminal of the sensor is disposed among two surfaces of the substrate.

The substrate may include a first surface and a second surface opposite to the first surface, at least one of a reference electrode, a working electrode, and a counter electrode may be disposed on the first surface and the second surface, and a plurality of connection terminals identical to the connection terminal may be disposed on at least one of the first surface and the second surface.

The substrate may include a first surface and a second surface opposite to the first surface and may be wound in a spiral shape such that the first surface faces outward and the second surface faces inward, and at least one of a reference electrode, a working electrode, and a counter electrode may be disposed on the first surface and the second surface.

The substrate may include a first surface and a second surface opposite to the first surface and may be wound in a spiral shape such that the first surface faces outward and the second surface faces inward, at least one reference electrode may be disposed on the first surface, and at least one working electrode and at least one counter electrode may be disposed on the second surface.

### Advantageous Effects

According to embodiments of the present invention, it is possible to obtain an in-body sensor having excellent sensing performance and a long lifetime by minimizing the influence of foreign materials. According to embodiments of the present invention, it is possible to obtain a sensing device including an in-body sensor capable of minimizing discomfort such as a feeling of irritation felt by a user.

### DESCRIPTION OF DRAWINGS

FIG. 1 illustrates a general continuous glucose monitoring system (CGMS).

FIG. 2 is a cross-sectional view of an example of a sensor in the CGMS of FIG. 1.

FIG. 3 is a block diagram of a sensing device according to one embodiment of the present invention.

FIG. 4A is a cross-sectional view of a sensor according to one embodiment of the present invention, and FIG. 4B is a top view of the sensor according to one embodiment of the present invention.

FIG. 5A is a top view of a sensor according to another embodiment of the present invention, FIG. 5B is a cross-sectional view along line A-A′ of FIG. 5A, and FIG. 5C is a cross-sectional view along line B-B′ of FIG. 5A.

FIG. 6A is a top view of a sensor according to still another embodiment of the present invention, FIG. 6B is a cross-sectional view along line A-A′ of FIG. 6A, and FIG. 6C is a cross-sectional view along line B-B′ of FIG. 6A.

FIG. 7A is a top view of a sensor according to yet another embodiment of the present invention, and FIG. 7B is a bottom view of the sensor according to yet another embodiment of the present invention.

FIG. 8 is a view illustrating a form in which a sensor is wound in a spiral shape according to one embodiment of the present invention.

FIG. 9 is a view illustrating a form in which a sensor is wound in a spiral shape according to another embodiment of the present invention.

FIG. 10 is a view for describing a principle of a sensor wound in a spiral shape.

FIG. 11 shows views for describing a process of manufacturing a sensor and a process of injecting the sensor into the body according to an embodiment of the present invention.

FIG. 12 is a top view of a stretchable substrate according to an embodiment of the present invention.

FIG. 13 is a cross-sectional view of a sensing device according to one embodiment of the present invention.

FIG. 14 is a cross-sectional view of a sensing device according to another embodiment of the present invention.

FIG. 15 is an image of implementing a sensing device according to one embodiment of the present invention.

### MODES OF THE INVENTION

Hereinafter, exemplary embodiments of the present invention will be described in detail with reference to the accompanying drawings.

However, the technical spirit of the present invention is not limited to the few embodiments disclosed below but can be implemented in various different forms. Without departing from the technical spirit of the present invention, one or more components may be selectively combined and substituted to be used between the embodiments.

Also, unless defined otherwise, terms (including technical and scientific terms) used herein may be interpreted as having the same meaning as commonly understood by one of ordinary skill in the art to which the present invention belongs. General terms like those defined in a dictionary may be interpreted in consideration of the contextual meaning of the related technology.

Furthermore, the terms used herein are intended to illustrate embodiments but are not intended to limit the present invention.

In the present specification, terms expressed in the singular form may include the plural form unless otherwise specified. When “at least one (or one or more) of A, B, and C” is expressed, it may include one or more of all possible combinations of A, B, and C.

In addition, terms such as “first,” “second,” “A,” “B,” “(a),” and “(b)” may be used herein to describe components of the embodiments of the present invention.

The terms are not used to define an essence, order, or sequence of a corresponding component but used merely to distinguish the corresponding component from other components.

In a case in which one component is described as being “connected,” “coupled,” or “joined” to another component, such a description may include both a case in which the one component is “connected,” “coupled,” and “joined” directly to the other component and a case in which the one component is “connected,” “coupled,” and “joined” to the other component with still another component disposed between the one component and the other component.

In addition, in a case in which any one component is described as being formed or disposed “on (or under)” another component, such a description includes both a case in which the two components are formed in direct contact with each other and a case in which the two components are in indirect contact with each other with one or more other components interposed between the two components. In addition, in a case in which one component is described as being formed “on (or under)” another component, such a description may include a case in which the one component is formed at an upper side or a lower side with respect to the other component.

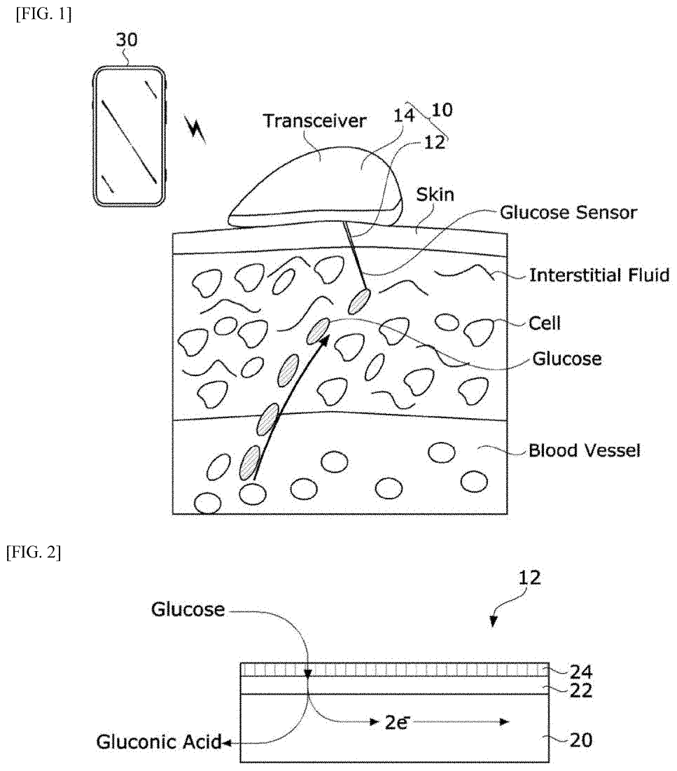

FIG. 1 illustrates a general continuous glucose monitoring system (CGMS). FIG. 2 is a cross-sectional view of an example of a sensor in the CGMS of FIG. 1.

Referring to FIGS. 1 and 2, a general CGMS 10 includes an in-body sensor 12 and a transmitter 14. The in-body sensor 12 may be in the form of a needle that penetrates skin and is inserted into the body. The in-body sensor 12 may include an electrode 20, an enzyme layer 22 disposed on the electrode 20, and a semi-permeable membrane 24 disposed on the enzyme layer 22. The CGMS 10 may be a system for measuring blood glucose, and the enzyme layer 22 may include glucose oxidase. When the in-body sensor 12 penetrates the skin and is inserted into the body, glucose in an interstitial fluid reacts with the glucose oxidase in the enzyme layer 22 and is converted into gluconic acid to release certain electric charges. The certain electric charges react with the electrode 20 to generate a current, and the current flowing in the electrode 20 is transmitted to the transmitter 14 outside the body along a wire (not shown). The transmitter 14 transmits data related to the current transmitted from the electrode 20 to an external terminal 30, and thus the external terminal 30 may output blood glucose information of the inside of the body.

Here, for convenience of description, an example of the general CGMS has been described, but embodiments of the present invention are not limited thereto. Embodiments of the present invention can be applied to various in-body sensors which penetrate into the body and detect body components in an interstitial fluid.

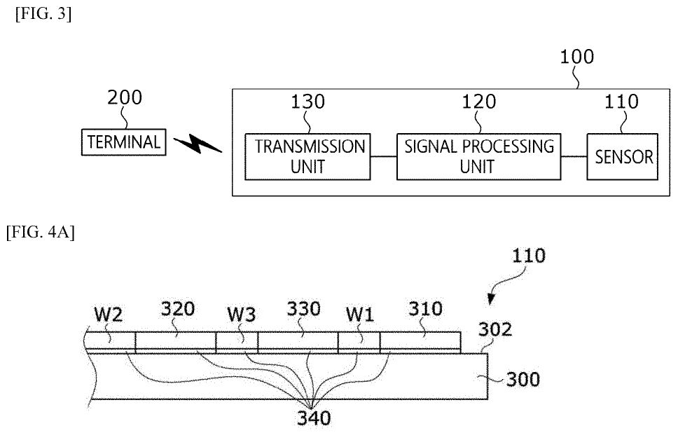

FIG. 3 is a block diagram of a sensing device according to one embodiment of the present invention.

Referring to FIG. 3, a sensing device 100 includes a sensor 110, a signal processing unit 120, and a transmission unit 130, and the transmission unit 130 communicates with an external terminal 200.

The sensor 110 penetrates skin and is inserted into the body to detect body components in an interstitial fluid. To this end, the sensor 110 may use an electrochemical reaction between a certain body component and a bioreactive material reacting therewith. When ions and/or electrons are generated through the electrochemical reaction between the certain body component and the bioreactive material reacting therewith, the presence or concentration of the certain body component may be detected using a current due to the generation of the ions and/or electrons. Since at least a portion of the sensor 110 is injected into the body, in the present specification, the sensor 110 may be referred to as an in-body sensor. A specific structure of the sensor 110 will be described below.

Here, the certain body component is not limited to blood glucose and may be any one of various biochemical materials or various biomarkers such as blood glucose, lactic acid, cholesterol, dopamine, coral, Na⁺, Ka⁺, and urea present in blood or interstitial fluid. The bioreactive material may be a material that reacts with the certain body component and may be an enzyme or the like. For example, when the sensor 110 is to detect a glucose level in the body, the bioreactive material may be glucose oxidase.

The sensor 110 includes a connection wire and a connection terminal, and an electrode of the sensor 110 is connected to the signal processing unit 120 through the connection wire and the connection terminal. Here, the connection wire may be connected to the electrode of the sensor 110, and a current flowing in the electrode of the sensor 110 disposed in the body may be transmitted to the signal processing unit 120 outside the body through the connection wire and the connection terminal. The signal processing unit 120 calculates information about the certain body component using an amount of the current received from the sensor 110. To this end, the signal processing unit 120 may perform analog-to-digital conversion on the amount of the current received from the sensor 110 and then may calculate a concentration of the certain body component.

The signal processing unit 120 transmits calculated information to the external terminal 200 through the transmission unit 130. In this case, the transmission unit 130 may communicate with the external terminal 200 in a wireless or wired manner, and the external terminal 200 may output the information received from the transmission unit 130 to a display or the like.

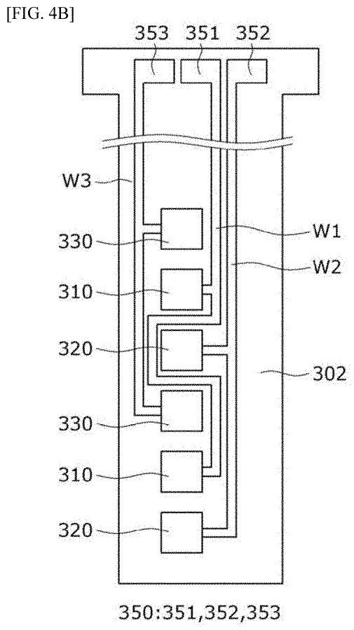

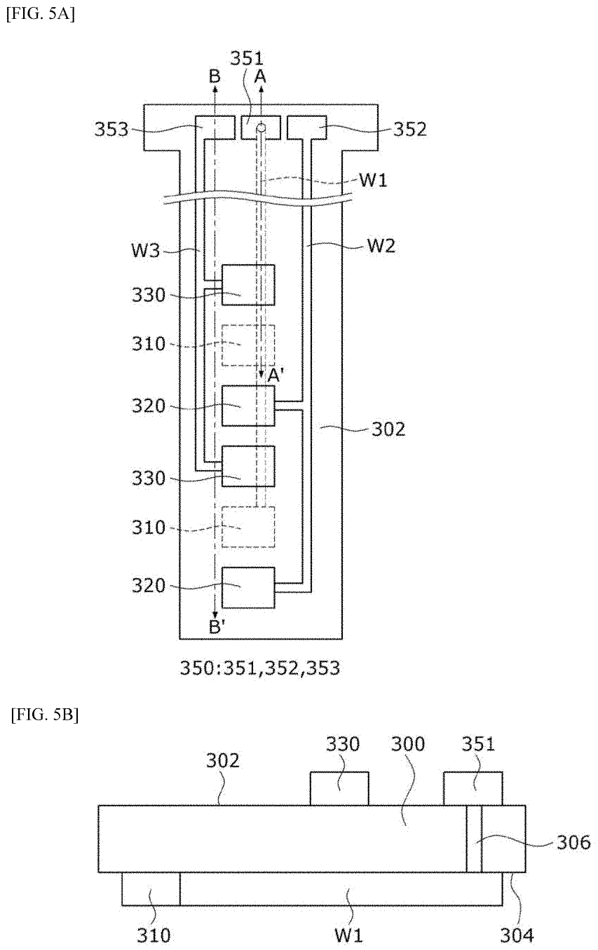

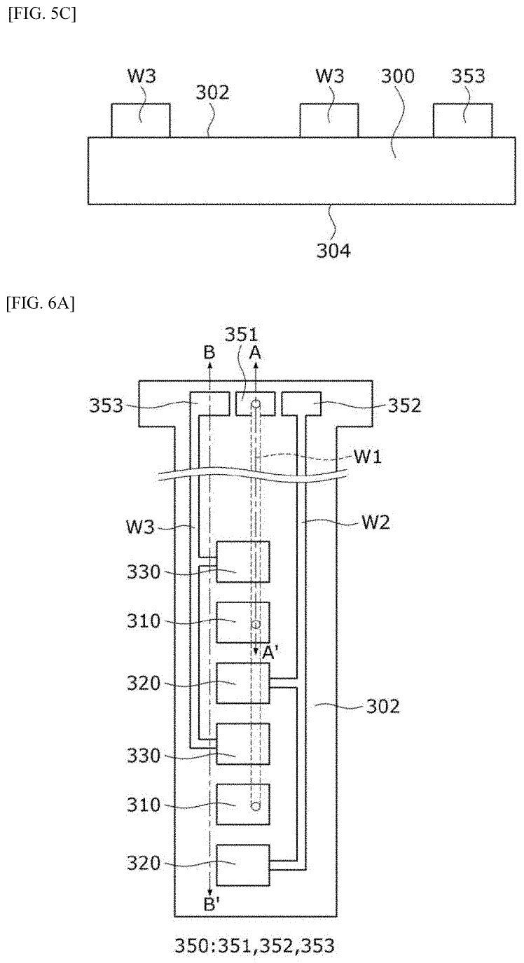

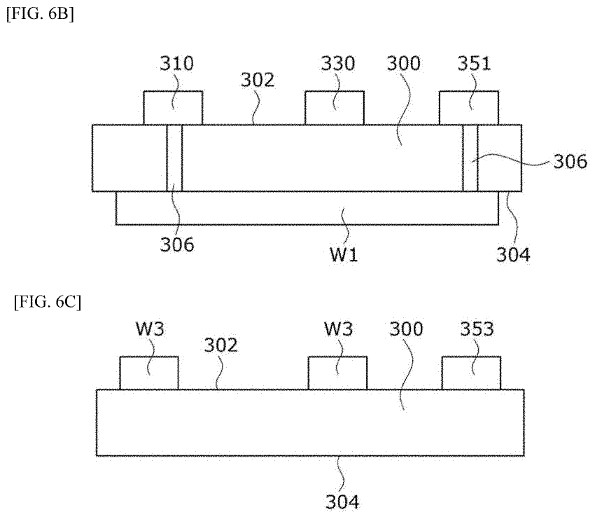

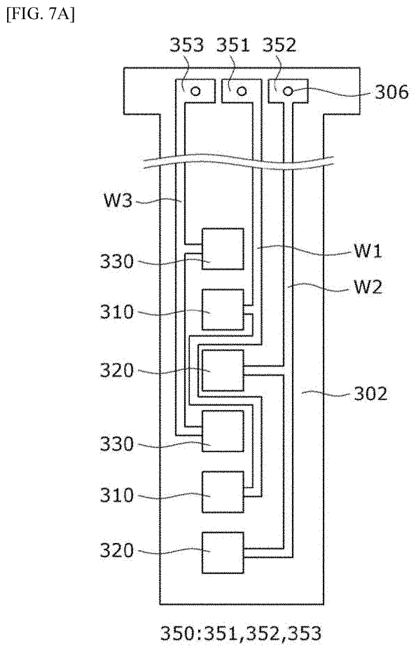

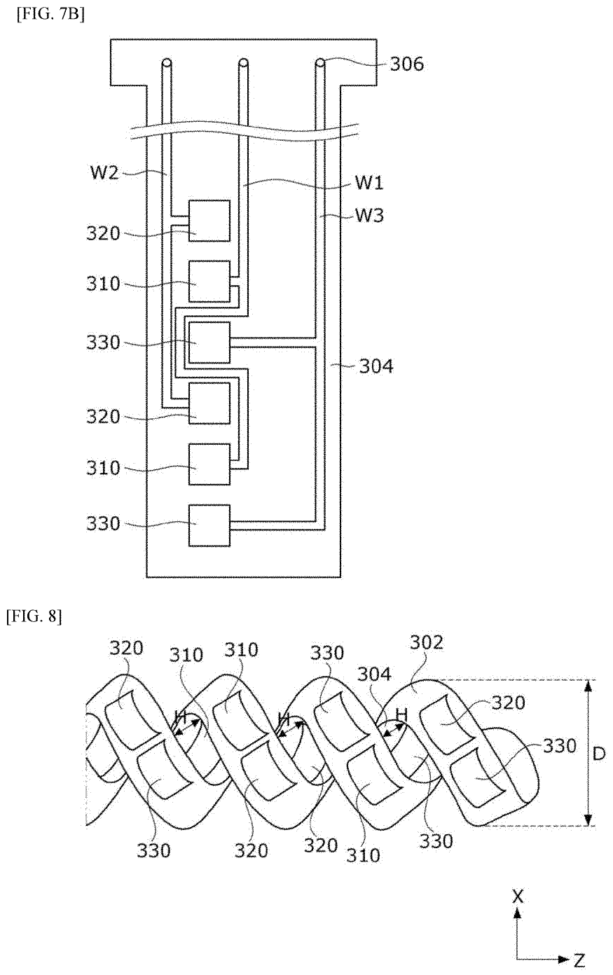

FIG. 4A is a cross-sectional view of a sensor according to one embodiment of the present invention, and FIG. 4B is a top view of the sensor according to one embodiment of the present invention. FIG. 5A is a top view of a sensor according to another embodiment of the present invention, FIG. 5B is a cross-sectional view along line A-A′ of FIG. 5A, and FIG. 5C is a cross-sectional view along line B-B′ of FIG. 5A. FIG. 6A is a top view of a sensor according to still another embodiment of the present invention, FIG. 6B is a cross-sectional view along line A-A′ of FIG. 6A, and FIG. 6C is a cross-sectional view along line B-B′ of FIG. 6A. FIG. 7A is a top view of a sensor according to yet another embodiment of the present invention, and FIG. 7B is a bottom view of the sensor according to yet another embodiment of the present invention.

Referring to FIGS. 4 to 7, a sensor 110 includes a substrate 300, and a reference electrode 310, a working electrode 320, and a counter electrode 330 which are disposed on the substrate 300.

Here, the substrate 300 may be flexible and may include a first surface 302 and a second surface 304 opposite to the first surface 302. Here, the substrate 300 of the sensor 110 may be a flexible substrate. The flexible substrate may be a flexible substrate that is unbreakable, bendable, rollable, and foldable. To this end, the substrate 300 may be made of, for example, a liquid crystal polymer (LCP), poly(ether ether ketone) (PEEK), polyimde (PI), or the like. Accordingly, since the substrate 300 is biocompatible and is flexibly bendable according to a flow of an interstitial fluid in the body, the substrate 300 may minimize a user's feeling of irritation and may be thermoformed. In addition, the substrate 300 may have a thickness of 10 μm to 150 μm, preferably a thickness of 30 μm to 130 μm, and more preferably a thickness of 50 μm to 100 μm. Accordingly, the shape of the thermoformed substrate 300 can be stably maintained.

The working electrode 320 may be an electrode at which an electrochemical reaction occurs and may be coated with a bioreactive material reacting with a certain body component. Here, the certain body component may be a component to be detected by the sensor 110 and may be any one of various biochemical materials or various biomarkers such as blood glucose, lactic acid, cholesterol, dopamine, coral, Na⁺, Ka⁺, and urea present in blood or interstitial fluid. The bioreactive material may be a material that reacts with the certain body component and may be an enzyme or the like. Although not shown, a semi-permeable membrane may be further disposed on the bioreactive material. Accordingly, only the certain body component to be detected can permeate through the semi-permeable membrane so that it is possible to prevent a problem of the bioreactive material applied on the working electrode 320 separating from the working electrode 320.

The reference electrode 310 is an electrode that forms a potential difference with the working electrode 320, and the counter electrode 330 is an electrode for measuring a current signal of the working electrode 320. That is, a constant voltage may be maintained in the counter electrode 330, and a current may flow in the working electrode 320 due to a reaction between the bioreactive material and the certain body component. The reference electrode 310 may serve to apply the constant voltage to the counter electrode 330. The working electrode 320 may be referred to as an operation electrode, and the counter electrode 330 may be referred to as a relative electrode.

Meanwhile, referring to FIGS. 4A and 4B, at least one reference electrode 310, at least one working electrode 320, and at least one counter electrode 330 may be disposed on the first surface 302 of the substrate 300, and the reference electrode 310, the working electrode 320, and the counter electrode 330 may be connected to connection terminals 351, 352, and 353 through wires W1, W2, and W3, respectively. Here, the wires W1, W2, and W3 and the connection terminals 351, 352, and 353 may transmit currents flowing in the electrodes 310, 320, and 330 of the sensor 110 to a signal processing unit 120 outside the body.

Alternatively, as shown in FIGS. 5A to 5C, at least one of the reference electrode 310, the working electrode 320, and the counter electrode 330 may be disposed on each of the first surface 302 and the second surface 304 of the substrate 300. For example, the working electrode 320 and the counter electrode 330 may be disposed on the first surface 302 of the substrate 300, and the reference electrode 310 may be disposed on the second surface 304. In this case, a connection terminal 351 connected to the reference electrode 310 may be disposed on the first surface 302 of the substrate 300, and a wire W1 connecting the reference electrode 310 and the connection terminal 351 may be disposed on the second surface 304 together with the reference electrode 310 to be connected to the connection terminal 351 through a via hole 306. Accordingly, since the reference electrode 310, the working electrode 320, and the counter electrode 330 are disposed on two surfaces of the substrate 300, a unit volume occupied by the sensor 110 or the number of electrodes disposed per unit area is increased, thereby increasing measurement precision.

Alternatively, as shown in FIGS. 6A to 6C, all of the reference electrode 310, the working electrode 320, the counter electrode 330, and connection terminals 350 may be disposed on one surface of the substrate 300, and some or all of the wires W1, W2, and W3 for connecting the reference electrode 310, the working electrode 320, and the counter electrode 330 to the connection terminals 350 may be disposed on the other surface thereof. For example, the reference electrode 310, the working electrode 320, the counter electrode 330, the connection terminals 350, and the wires W2 and W3 may be disposed on the first surface 302 of the substrate 300, and the wire W1 for connecting the reference electrode 310 and a connection terminal 351 may be disposed on the second surface 304 of the substrate 300. Accordingly, since the electrodes 310, 320, and 330 and the wires W1, W2, and W3 are distributed and disposed on two surfaces of the substrate 300, a unit volume occupied by the sensor 110 or the number of electrodes disposed per unit area is increased, thereby increasing measurement precision.

To this end, at least one via hole 306 may be formed in the substrate 300, at least one of the wire W1 connected to the reference electrode 310, the wire W2 connected to the working electrode 320, and the wire W3 connected to the counter electrode 330 may pass through the via hole 306, and the reference electrode 310, the working electrode 320, and the counter electrode 330 may be connected to the connection terminals 351, 352, and 353 through the wires W1, W2, and W3, respectively. Here, currents flowing in the wires W1, W2, and W3 and the connection terminals 351, 352, and 353 may be transmitted to a signal processing unit 120 outside the body. Accordingly, since the connection terminals 350 are gathered on one side of two surfaces of the substrate 300 and then drawn out of the body, wiring is easy.

Alternatively, as shown in FIGS. 7A and 7B, a plurality of reference electrodes 310, a plurality of working electrode 320, and a plurality of counter electrodes 330 may be disposed on each of the first surface 302 and the second surface 304 of the substrate 300. Accordingly, since the plurality of reference electrodes 310, the plurality of working electrodes 320, and the plurality of counter electrodes 330 are disposed on two surfaces of the substrate 300, a unit volume occupied by the sensor 110 or the number of electrodes disposed per unit area is increased, thereby increasing measurement precision.

When connection terminals 350 are disposed on the first surface 302 of the substrate 300, wires W1, W2, and W3 connected to the electrodes 310, 320, and 330 disposed on the second surface 304 of the substrate 300 may be connected to the connection terminals 350 disposed on the first surface 302 of the substrate 300 through via holes 306.

Although not shown, one set of the reference electrode 310, the working electrode 320, and the counter electrode 330 may be connected to one set of the connection terminals 351, 352, and 353 through one set of the wires W1, W2, and W3. That is, when one sensor 110 includes a plurality of sets of the reference electrode 310, the operation electrode 320, and the counter electrode 330, a set of the connection terminals 351, 352, and 353 may be individually disposed for each set of the reference electrode 310, the working electrode 320, and the counter electrode 330. Accordingly, sensing accuracy can be increased.

Meanwhile, referring to FIG. 4A, seed layers 340 may be further disposed between the substrate 300, and the reference electrode 310, the working electrode 320, and the counter electrode 330, and the seed layer 340 may include at least one selected from titanium (Ti) and nickel (Ni). Accordingly, bonding strength between the substrate 300, and the reference electrode 310, the working electrode 320, and the counter electrode 330 can be improved.

Alternatively, before the seed layer 340 is formed on the substrate 300, the substrate 300 may be preprocessed. For example, when a surface of the substrate 300 is plasma-processed or is coated with a hydrophilic primer, since the surface of the substrate 300 becomes hydrophilic, the seed layer 340 is advantageously formed on the surface of the substrate 300.

Accordingly, adhesion between the substrate 300, the seed layer 34, and the electrodes 310, 320, and 330 can be improved.

Meanwhile, each of the reference electrode 310, the working electrode 320, and the counter electrode 330 may include nanoparticles of at least one selected from gold (Au) and platinum (Pt), the reference electrode 310 may further include silver chloride (AgCl), and each of the reference electrode 310, the working electrode 320, and the counter electrode 330 may be disposed on the substrate 300 through deposition, sputtering, plating, evaporation, coating, or the like. A particle size of the nanoparticles constituting the electrodes 310, 320, and 330 may be changed according to processing conditions of deposition, sputtering, plating, evaporation, coating, or the like. According to an embodiment of the present invention, each of the reference electrode 310, the working electrode 320, and the counter electrode 330 may include at least one selected from gold (Au) and platinum (Pt). In this case, each of the reference electrode 310, the working electrode 320, and the counter electrode 330 may be made of a wrinkled metal or a porous metal. Accordingly, sensing accuracy can be increased. In this case, each of the reference electrode 310, the working electrode 320, and the counter electrode 330 may be made of nanoparticles having a D50 of 5 nm to 100 nm, preferably a D50 of 5 nm to 75 nm, and more preferably a D50 of 5 nm to 50 nm. Accordingly, since surfaces of the electrodes 310, 320, and 330 are smooth, the possibility of adsorption of foreign materials can be reduced. Here, foreign materials may be materials such as proteins, platelets, cells, fibroblasts, immune substances, or blood cells present in blood or interstitial fluid other than body components to be detected. When foreign materials are adsorbed onto the surfaces of the electrodes 310, 320, and 330, a sensing function may be degraded, and a lifetime of a sensor may be shortened.

Alternatively, in order to reduce the possibility of foreign materials being adsorbed onto the electrodes 310, 320, and 330, the surfaces of the electrodes 310, 320, and 330 may be coated with a hydrophobic material. When the surfaces of the electrodes 310, 320, and 330 are coated with the hydrophobic material, foreign materials may not be adsorbed onto the surfaces of the electrodes 310, 320, and 330. Here, the hydrophobic material may be a biocompatible hydrophobic material, and a type thereof is not particularly limited.

Meanwhile, according to an embodiment of the present invention, a sensor may be implemented in a form wound in a spiral shape.

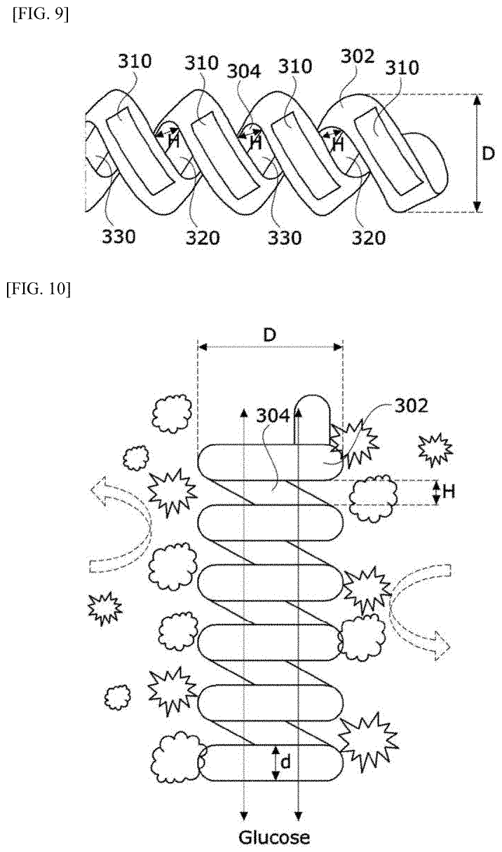

FIG. 8 is a view illustrating a form in which a sensor is wound in a spiral shape according to one embodiment of the present invention, and FIG. 9 is a view illustrating a form in which a sensor is wound in a spiral shape according to another embodiment of the present invention.

Referring to FIGS. 8 to 9, a substrate 300 may be wound in a spiral shape such that a first surface 302 of the substrate 300 faces outward and a second surface 304 thereof faces inward. Here, the spiral shape may be a three-dimensional shape that repeatedly rotates with a certain curvature and extends in a certain direction (for example, a Z direction) and may be a shape in which the substrate continues around an outer circumferential surface of a cylinder. The spiral shape may be used interchangeably with a helical shape or the like. When the substrate 300 is wound in a spiral shape as described above, since stress applied to the substrate 300 may be distributed, the substrate 300 may be more flexible than a flat substrate, thereby reducing an effect on a flow of interstitial fluid and reducing a feeling of irritation.

In this case, the substrate 300 may be wound in a spiral shape having a width D that is in a range of 10 μm to 1,000 μm, preferably a range of 100 μm to 800 μm, and more preferably a range of 300 μm to 600 μm. The width D may refer to a length in an X direction perpendicular to the Z direction in a spiral shape extending in the Z direction and may refer to a maximum distance between the first surface 302 and another first surface 302 at a certain position on a Z axis. When the width D of the substrate 300 satisfies such a numerical range, a certain body component may freely pass through an empty space formed by the second surface 304. A capillary action may act on the empty space formed by the second surface 304, and an interstitial fluid may be easily collected and discharged.

In addition, a gap H between spirals constituting the spiral shape of the substrate 300 may be in a range of 1 μm to 300 μm, preferably a range of 5 μm to 200 μm, and more preferably a range of 10 μm to 100 μm. When the gap H between the spirals satisfies such a numerical range, it is possible to reduce a possibility of foreign materials such as proteins penetrating into the spiral shape, that is, the empty space formed by the second surface 304.

Meanwhile, according to one embodiment of the present invention, as shown in FIG. 8, a reference electrode 310, a working electrode 320, and a counter electrode 330 may be disposed on the first surface 302 of the substrate 300, and a reference electrode 310, a working electrode 320, and a counter electrode 330 may also be disposed on the second surface 304 of the substrate 300. When the reference electrode 310, the working electrode 320, and the counter electrode 330 are disposed on two surfaces of the substrate 300, a contact area with a certain component to be detected is increased, thereby increasing sensing accuracy.

Alternatively, according to another embodiment of the present invention, as shown in FIG. 9, the reference electrode 310 may be disposed on the first surface 302 of the substrate 300, and the working electrode 320 and the counter electrode 330 may be disposed on the second surface 304. Here, the substrate 300 may be wound in a spiral shape such that the first surface 302 on which the reference electrode 310 is disposed faces outward, and the second surface 304 on which the working electrode 320 and the counter electrode 330 are disposed faces inward. As shown in FIG. 10, when the substrate 300 is wound in a spiral shape having a width D that is in a range of 10 μm to 1,000 μm, preferably a range of 100 μm to 800 μm, and more preferably a range of 300 μm to 600 μm and having a gap H that is in a range of 1 μm to 300 μm, preferably a range of 5 μm to 200 μm, and more preferably a range of 10 μm to 100 μm, a certain body component to be detected can freely pass through the interior of the spiral shape, that is, an empty space formed by the second surface 304, and a possibility of penetration of foreign materials can be reduced. According to an embodiment of the present invention, when the first surface 302, on which the reference electrode 310 that has less effect on the degradation of a sensing function is disposed, is disposed to face outward, and the second surface 304, on which the working electrode 320 and the counter electrode 330, at which an electrochemical reaction substantially occurs, are disposed, is disposed to face inward, it is possible to improve the accuracy and durability of a sensor.

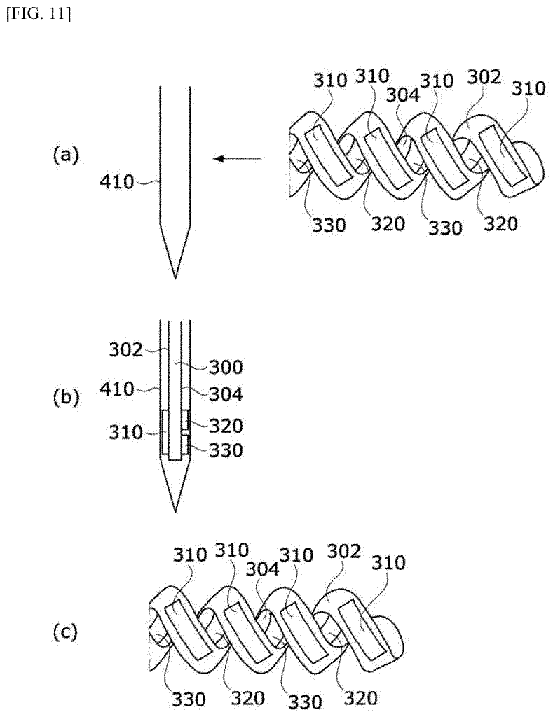

FIG. 11 shows views for describing a process of manufacturing a sensor and a process of injecting the sensor into the body according to an embodiment of the present invention. Since the sensor is injected into the body, the sensor may be referred to as an in-body sensor in the present specification.

In order to manufacture the sensor according to the embodiment of the present invention, electrodes 310, 320, and 330 are formed on a substrate 300. Here, the substrate 300 may be made of an LCP, PEEK, PI, or the like. As described above, after a surface of the substrate is preprocessed through plasma processing or coating using a hydrophilic primer, a seed layer may be formed to form the electrodes. As described above, the electrodes 310, 320, and 330 may be formed through a method of depositing, sputtering, plating, evaporating, or applying nanoparticles of at least one selected from gold (Au) and platinum (Pt). The surface preprocessing of the substrate 300, the formation of the seed layer, and the formation of the electrodes may all be performed on two surfaces of the substrate.

Next, the substrate on which the electrodes 310, 320, and 330 are formed is thermoformed. Accordingly, the substrate on which the electrodes are formed may be wound in a spiral shape.

Next, the electrodes are coated with an enzyme. To this end, dip casting may be performed on the substrate, on which the electrodes are formed and which is wound in the spiral shape, in an enzyme solution, an enzyme solution may be sprayed onto the electrodes, or after the substrate on which the electrodes are formed is spread and fixed, drop casting may be performed thereon in an enzyme solution. As described above, after the substrate on which the electrodes are formed is thermoformed, when the electrodes are coated with an enzyme, it is possible to prevent a problem of the enzyme being denatured by heat.

Next, the sensor coated with the enzyme is inserted into a sensor guide.

Referring to FIG. 11A, a sensor guide 400 has a needle shape with a sharp end, and an empty space may be formed in an interior 410 thereof Although not shown, the end of the sensor guide 400 may be open. The sensor formed according to the above-described method is inserted into the sensor guide 400. In this case, the sensor may be thermoformed and wound in a spiral shape or may be inserted into the sensor guide 400 in a spread planar shape.

As shown in FIG. 11B, the sensor spread in a planar shape is surrounded by the sensor guide 400 and is injected into the body together with the sensor guide 400.

Thereafter, when the sensor guide 400 is independently drawn out from the body, the sensor may be separated from the sensor guide 400 and may be wound again in a thermoformed shape.

Alternatively, the sensor guide 400 may be made of a biodegradable material. Here, the biodegradable material may be a biodegradable polymer, and the biodegradable polymer may be, for example, polylactide (PLA) or a polyglycolic acid (PGA)-based polymer.

Accordingly, after the sensor is injected into the body together with the sensor guide 400, when the sensor guide 400 is biodegraded in the body, the sensor may be wound again in a thermoformed shape.

As described above, since the substrate 300 according to the embodiment of the present invention is made of an LCP, PEEK, or PI, the substrate 300 may be formed into a spiral shape by heat, after the substrate 300 is inserted into the sensor guide 400 in a state in which it is spread by a physical force and injected into the body, when the substrate 300 is separated from the sensor guide 400 or the sensor guide 400 is decomposed, the substrate 300 may be restored to a spiral shape again.

Accordingly, it is easy to inject the sensor twisted in a spiral shape into the body without causing discomfort to a user or damage to the sensor.

Meanwhile, as described above, a current of an in-body sensor, that is, a current of a sensor 110, may be transmitted to a signal processing unit 130 outside the body through a connection unit 120, and the signal processing unit 130 may calculate information about a certain body component using an amount of the current received from the sensor 110 through the connection unit 120 and may transmit the calculated information to an external terminal 200 through a transmission unit 140. The transmitter 14 of FIG. 1 may include a portion of the connection unit 120, the signal processing unit 130, and the transmission unit 140 and may be attached to the outside of the body, generally, to user's skin.

In an embodiment of the present invention, the transmitter 14 of FIG. 1 is to be implemented using a stretchable substrate. When the transmitter 14 is implemented using the stretchable substrate, the transmitter 14 is stretchable and directly attached to the skin, thereby minimizing a feeling of irritation or discomfort felt by a user.

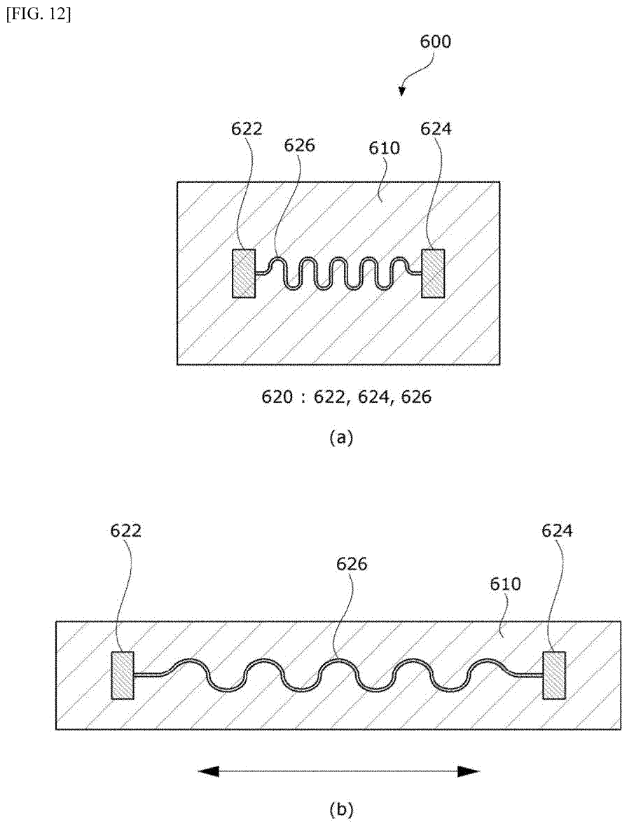

FIG. 12 is a top view of a stretchable substrate according to an embodiment of the present invention.

Referring to FIG. 12, a stretchable substrate 600 includes a base 610 and a line 620 disposed on the base 610.

The line 620 includes a first pad 622, a second pad 624, and a connector 626 for connecting the first pad 622 and the second pad 624.

Here, the base 610 may have a flexible property of being unbreakable, bendable, rollable, and foldable and may further have a property of being stretchable or contractible. Accordingly, the base 610 may be implemented to have a curved surface, may be stretched in at least one direction by an external force, and may be restored to the original state thereof when the external force is removed. Accordingly, the base 610 may be a stretchable base. To this end, the base 610 may include a polymer resin having certain elasticity. For example, the base 610 may include at least one selected from polyurethane (PU) and polydimethylsiloxane (PDMS). Accordingly, the base 610 can be elastically stretched or contacted according to an external force.

Meanwhile, the first pad 622 and the second pad 624 may be disposed on the base 610. The first pad 622 and the second pad 624 may be made of the same material as the connector 626 or may be made of a material that is different from that of the connector 626 and has conductivity. A semiconductor element is disposed on the first pad 622 and the second pad 624, and the first pad 622 and the second pad 624 may be connected to the semiconductor element. Alternatively, the first pad 622 and the second pad 624 may be electrically connected to components of the base 610 or may be connected to an external power source. In this case, the first pad 622 and the second pad 624 may be bent or stretched along with bending or stretching/contraction of the base 610.

The first pad 622, the second pad 624, and the connector 626 may include a support layer and a metal layer disposed on the support layer. The metal layer may include at least one selected from among gold (Au), copper (Cu), platinum (Pt), and silver (Ag), and the support layer may include at least one selected from among an LCP, PEEK, and PI. In this case, the support layer may be disposed in contact with the base 610. Accordingly, an adhesive force between the metal layer and the base 610 can be increased.

Meanwhile, the connector 626 may include a repeated curved pattern. For example, the repeated curved pattern may be a meandering pattern or the like that is meandering. Accordingly, the line 620 may be a stretchable line, and as shown in FIG. 12B, the connector 626 may also be stretched or contracted as the stretchable substrate 610 is stretched or contracted.

Alternatively, the connector 626 may be wound in a spiral shape between the first pad 622 and the second pad 624. The spiral shape may be a three-dimensional shape that repeatedly rotates with a certain curvature and extends in a certain direction, for example, a direction parallel to a plane direction of the base 610, that is, a direction from the first pad 622 toward the second pad 624 or a direction from the second pad 624 toward the first pad 622. The spiral shape may be used interchangeably with a helical shape or the like. In this case, a diameter of the spiral shape may be in a range of 30 μm to 1 mm, preferably a range of 50 μm to 500 μm, and more preferably a range of 100 μm to 300 μm, and a gap between spirals of the spiral shape may be in a range of 1 μm to 5 mm, preferably a range of 100 μm to 3 mm, and more preferably a range of 300 μm to 2 mm.

Accordingly, the line 620 may also be bent or stretched/contracted without restriction as the base 610 is bent or stretched/contracted, and a degree of integration of the line 620 may be increased so that the overall size of the stretchable substrate 600 can be miniaturized. In particular, even when the line 620 is made of an inorganic material that has no elasticity, since the line 620 may be bent or stretched/contracted together with the base 610 due to the spiral shape, the line 620 may not be restricted by a material. In addition, even when the line 620 is also bent or stretched/contracted as the base 610 is bent or stretched/contracted, since an actual length of the line 620 is not increased, a change in resistance can be minimized, and a reliable stretchable substrate can be obtained.

According to an embodiment of the present invention, a sensor may be connected to a stretchable substrate, and the stretchable substrate may include functions of a signal processing unit and a transmission unit. According to an embodiment of the present invention, the stretchable substrate included in a sensing device may be stretched up to 30% to 50% by an external force.

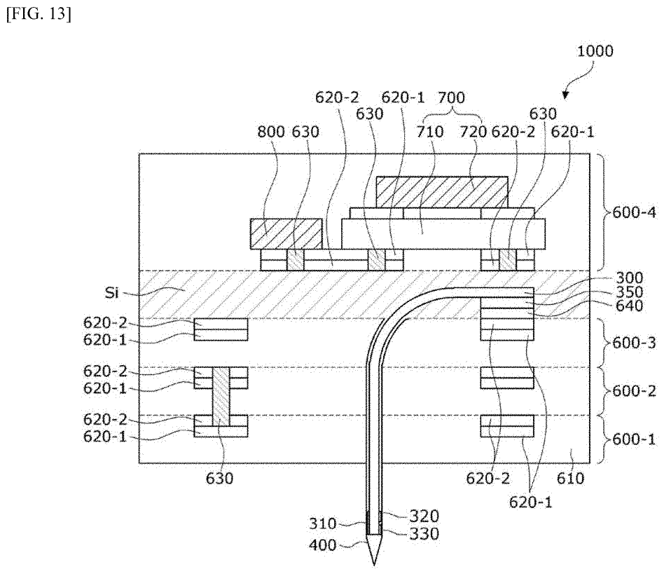

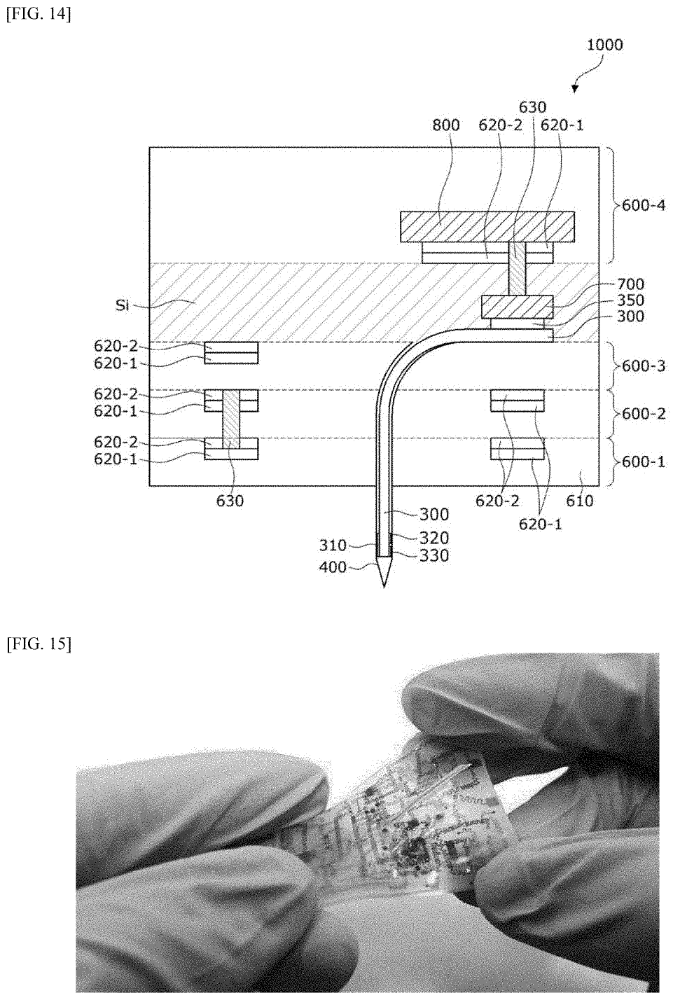

FIG. 13 is a cross-sectional view of a sensing device according to one embodiment of the present invention, and FIG. 14 is a cross-sectional view of a sensing device according to another embodiment of the present invention.

Referring to FIGS. 13 to 14, a sensing device 1000 includes a sensor and a stretchable substrate connected to the sensor. Here, the sensor may be the sensor described with reference to FIGS. 4 to 11.

As described above, each stretchable substrate includes a base and a line disposed on the base.

As an example, each of stretchable substrates 600-1, 600-2, 600-3, and 600-4 may be manufactured through a method in which, after a metal layer 620-2 and a support layer 620-1 are sequentially applied on a polyethylene terephthalate (PET) film, the metal layer 620-2 and the support layer 620-1 are patterned to form lines, and then the lines are buried in the base 610, and the PET film is delaminated.

The stretchable substrates 600-1, 600-2, 600-3, and 600-4 manufactured through the method may be stacked as a plurality of layers and thus may be stacked as a plurality of line layers. Lines disposed on different layers may be electrically connected to each other through via holes 630 formed in the lines.

For example, in two layers of lines included in different stretchable substrates 600-1, 600-2, 600-3, and 600-4, the via holes 630 may be formed and then may be filled with a conductive material to form vias. The conductive material filling the via holes may be any one material selected from among copper (Cu), silver (Ag), tin (Sn), gold (Au), nickel (Ni), and palladium (Pd) and may fill the via holes through any one of electroless plating, electrolytic plating, screen printing, sputtering, evaporation, ink jetting, and dispensing, or a combination method thereof. Vias may be formed by forming a seed layer through electroless plating using palladium/nickel/chromium and then filling the via holes 630 with a metal material through electrolytic plating, screen printing, or the like. In addition, after a plurality of line layers such as first, second, third, and fourth layers are stacked, a via connecting the plurality of layers may be formed.

Although an example in which the sensing device includes a total of four layers of the stretchable substrates 600-1, 600-2, 600-3, and 600-4 and includes a total of four line layers by forming the line layer on each stretchable substrate is described here, the present invention is not limited thereto. The sensing device may include a total of two or more layers of the stretchable substrates and preferably a total of three or more layers of the stretchable substrates and thus may include a total of two or more line layers and preferably a total of three or more line layers. According to an embodiment of the present invention, a total thickness of the total of four layers of the stretchable substrates 600-1, 600-2, 600-3, and 600-4 may be 2 mm or less. Accordingly, it is possible to minimize a feeling of irritation and discomfort felt by a user.

Referring to FIGS. 13 and 14 as an example, a connection terminal 350 of the sensor may be disposed between the line layer of a third layer stretchable substrate 600-3 and the line layer of a fourth layer stretchable substrate 600-4, at least a portion of the sensor may be inserted into the stretchable substrates 600-1, 600-2, 600-3, and 600-4, and the remaining portion thereof may be drawn out of the stretchable substrates 600-1, 600-2, 600-3, and 600-4. For example, electrodes 310, 320, and 330 of the sensor may pass through some stretchable substrates 600-1, 600-2, and 600-3 of the plurality of stretchable substrates and may be drawn out of the stretchable substrates.

In this case, one surface of two surfaces of a substrate 300 of the sensor may be disposed to face the fourth layer stretchable substrate 600-4, and the other surface thereof may be disposed to face the third layer stretchable substrate 600-3.

A space, which is separated between the third layer stretchable substrate 600-3 and the fourth layer stretchable substrate 600-4 to arrange the sensor, may be filled with silicon (Si) or a silicone resin.

The connection terminal 350 of the sensor may be disposed between the third layer stretchable substrate 600-3 and the fourth layer stretchable substrate 600-4, the substrate 300 of the sensor on which wires are wired may pass through a total of three layers of the stretchable substrates 600-1, 600-2, and 600-3 and may be drawn out of the stretchable substrates 600-1, 600-2, and 600-3, and the electrodes 310, 320, and 330 of the sensor may be inserted into the body while accommodated in a sensor guide 400. In this case, a portion of the sensor guide 400 may be inserted into the stretchable substrates 600-1, 600-2, and 600-3, and the remaining portion of the sensor guide 400 may be exposed to the outside of the stretchable substrates 600-1, 600-2, and 600-3. Accordingly, the electrodes 310, 320, and 330 of the sensor may be stably fixed to the stretchable substrates 600-1, 600-2, and 600-3. For example, a length of the sensor guide 400 exposed to the outside of the stretchable substrates 600-1, 600-2, and 600-3 may be greater than a length of the sensor guide 400 inserted into the stretchable substrates 600-1, 600-2, and 600-3. Accordingly, the electrodes 310, 320, and 330 of the sensor may be inserted into the body so that a contact area with an interstitial fluid may be increased.

The connection terminal 350 of the sensor may be connected to a signal processing unit, and the signal processing unit may signal-process an amount of current received from the electrodes 310, 320, and 330 of the sensor through the connection terminal 350. The signal processing unit may be connected to a transmission unit, and a signal processed by the signal processing unit may be transmitted to the outside through the transmission unit.

To this end, as shown in FIG. 13, the connection terminal 350 of the sensor may be connected to the lines 620-1 and 620-2 of the stretchable substrate 600-3, and the lines 620-1 and 620-2 may be connected directly or indirectly to the signal processing unit. As shown in FIGS. 4B, 5A, 6A, 7A, and 7B, in the substrate 300 of the sensor 110, a width of a connection terminal region in which the connection terminal 350 is disposed may be greater than a width of an electrode region in which the electrodes 310, 320, and 330 are disposed. For example, in the substrate 300 of the sensor 110, the width of the connection terminal region in which the connection terminal 350 is disposed may be greater than 1 times and not more than 5 times the width of the electrode region in which the electrodes 310, 320, and 330 are disposed, may be preferably not less than 1.5 times and not more than 4 times the width of the electrode region, and may be more preferably not less than 2 times and not more than 3.5 times the width of the electrode region. Accordingly, the connection terminal can be stably bonded to the stretchable substrate 600-3, and when the sensor is injected into the body, the separation of the sensor can be prevented. In the substrate 300 of the sensor 110, when the width of the connection terminal region in which the connection terminal 350 is disposed is greater than 5 times the width of the electrode region in which the electrodes 310, 320, and 330 are disposed, since an area of the non-stretchable substrate 300 is too wide, a user may feel a feeling of irritation.

As described above, a line 620 may include a plurality of pads and a connector for connecting the plurality of pads, and the connection terminal 350 of the sensor 110 may be disposed between two line layers among a plurality of line layers as shown in FIG. 13. In this case, the connection terminal 350 may be bonded to a pad of the line 620-2 through an adhesive portion 640, the adhesive portion 640 may be a solder ball or a plating layer, and the plating layer may include at least one selected from among gold (Au), silver (Ag), copper (Cu), nickel (Ni), palladium (Pd), and chromium (Cr). According to an embodiment of the present invention, a width of the connection terminal 350 may be different from a width of the pad of the line 620-2. For example, a width of the adhesive portion 640 may be between the width of the connection terminal 350 and the width of the pad of the line 620-2.

As shown in FIG. 13, chips 700 and 800 implementing a signal processing unit 120 (see FIG. 3) and a transmission unit 130 (see FIG. 3) may be disposed on one of the stretchable substrates 600-1, 600-2, 600-3, and 600-4 stacked as the plurality of layers, for example, the fourth layer stretchable substrate 600-4.

That is, the signal processing unit and the transmission unit may be electrically connected to a layer of the line 620-2 to which the connection terminal 350 is connected.

Here, a signal processing unit 700 and a transmission unit 800 may be chips implemented as integrated circuits. In this case, the signal processing unit 700 may include a signal processing chip 720 disposed on a hard printed circuit board (PCB) 710. Accordingly, even when the stretchable substrate 600-4 is bent or stretched/contracted by an external force, it is possible to minimize a problem of the signal processing chip 720 being damaged by the bending or stretching/contracting of the stretchable substrate 600-4. Similarly, the transmission unit 800 may also be implemented in a form including a transmission chip disposed on a hard PCB.

Alternatively, as shown in FIG. 14, the signal processing unit 700 may be formed on the substrate 300 of the sensor. For example, a signal processing circuit pattern for processing a signal received from the electrodes through the connection terminal 350 may be further disposed on a surface on which the connection terminal 350 of the sensor is disposed among two surfaces of the substrate 300. That is, the signal processing unit 700 may be implemented in the form of a flexible PCB (FPCB) on the substrate 300 of the sensor. The signal processing unit 700 may be connected to the transmission unit 800 disposed on the fourth layer stretchable substrate 600-4. Accordingly, the signal processing unit 700 and the sensor can be easily connected.

FIG. 15 is an image of implementing a sensing device according to one embodiment of the present invention.

Referring to FIG. 15, it can be seen that the sensing device according to the embodiment of the present invention includes a sensor and a stretchable substrate, a connection terminal of the sensor is connected to the stretchable substrate, and an electrode of the sensor is disposed outside the stretchable substrate together with a sensor guide.

Although the present invention has been described with reference to the exemplary embodiments, it will be understood by those skilled in the art that various modifications and changes can be made in the present invention without departing from the spirit and scope of the present invention as defined in the appended claims.

1. A sensing device comprising:
a sensor which includes a substrate, an electrode disposed on the substrate, and a connection terminal disposed on the substrate and connected to the electrode; and
a stretchable substrate which is connected to the sensor and includes a base and a line disposed on the base,
wherein the connection terminal of the sensor is connected to the line of the stretchable substrate.

2. The sensing device of claim 1, wherein:
the line includes a plurality of line layers which are stacked; and
the connection terminal of the sensor is disposed between two line layers among the plurality of line layers.

3. The sensing device of claim 2, wherein the base is disposed between adjacent line layers among the plurality of line layers.

4. The sensing device of claim 2, wherein each of the plurality of line layers includes a metal layer and a support layer.

5. The sensing device of claim 1, wherein at least a portion of the sensor is inserted into the stretchable substrate, and a remaining portion thereof is drawn out of the stretchable substrate.

6. The sensing device of claim 5, wherein the electrode of the sensor is drawn out of the stretchable substrate, is accommodated in a biodegradable sensor guide, and is injected into a body together with the biodegradable sensor guide.

7. The sensing device of claim 6, wherein a portion of the biodegradable sensor guide is inserted into the stretchable substrate, and a remaining portion of the biodegradable sensor guide is exposed out of the stretchable substrate.

8. The sensing device of claim 1, wherein:
the substrate is divided into an electrode region in which the electrode is disposed and a connection terminal region in which the connection terminal is disposed; and
a width of the connection terminal region is greater than a width of the electrode region.

9. The sensing device of claim 1, wherein:
the line of the stretchable substrate includes a plurality of pads and a connector configured to connect the plurality of pads; and
a width of the connection terminal is different from a width of the pad.

10. The sensing device of claim 9, further comprising an adhesive portion disposed between the connection terminal and the pad.

11. The sensing device of claim 5, wherein:
the plurality of line layers include a first line layer facing a first surface on which the connection terminal of the sensor is disposed among two surfaces of the substrate and a second line layer facing a second surface opposite to the first surface; and
the sensing device further includes at least one of a signal processing unit and a transmission unit connected to the first line layer and buried in the stretchable substrate.

12. The sensing device according to claim 2, wherein a signal processing circuit pattern configured to process a signal received from the electrode through the connection terminal is further disposed on a first surface on which the connection terminal of the sensor is disposed among two surfaces of the substrate.

13. The sensing device of claim 2, wherein:
the substrate includes a first surface and a second surface opposite to the first surface;
at least one of a reference electrode, a working electrode, and a counter electrode is disposed on the first surface and the second surface; and
a plurality of connection terminals identical to the connection terminal are disposed on at least one of the first surface and the second surface.

14. The sensing device of claim 2, wherein:
the substrate includes a first surface and a second surface opposite to the first surface and is wound in a spiral shape such that the first surface faces outward and the second surface faces inward; and
at least one of a reference electrode, a working electrode, and a counter electrode is disposed on the first surface and the second surface.

15. The sensing device of claim 2, wherein:
the substrate includes a first surface and a second surface opposite to the first surface and is wound in a spiral shape such that the first surface faces outward and the second surface faces inward;
at least one reference electrode is disposed on the first surface; and
at least one working electrode and at least one counter electrode are disposed on the second surface.

16. The sensing device of claim 1, wherein the substrate is flexible.

17. The sensing device of claim 1, wherein the base is stretchable.

18. The sensing device of claim 4, wherein the metal layer includes at least one of Au, Cu, Pt and Ag and the support layer includes at least one of LCP(liquid crystal polymer), PEEK(poly ether ether ketone) and PI(polyimde).

19. The sensing device of claim 9, wherein the connector includes a repeated curved pattern.

20. The sensing device of claim 11, wherein at least one of the signal processing unit and the transmission unit includes a hard PCB and a chip disposed on the hard PCB.
